(19) United States
(12) Patent Application Publication
(10) Pub. No.: US 2026/0321158 A1
(43) Pub. Date: Oct. 15, 2026

(54) SURGICAL SIMULATOR, KIT AND SYSTEM FOR TRAINING AND PREOPERATIVE PLANNING OF A SURGICAL OPERATION

(71) Applicants: Politecnico di Milano, Milano (IT); Medizinische Universitaet Wien, Wien (AT)

(72) Inventors: Lorenzo Civilla, Wien (AT); Francesco Moscato, Wien (AT); Philippe Dodier, Wien (AT); Ewald Unger, Wien (AT); Alberto Cesare Luigi Redaelli, Milano (IT); Maria Chiara Palumbo, Milano (IT); Markus Koenigshofer, Wien (AT)

(73) Assignees: Politecnico di Milano, Milano (IT); Medizinische Universitaet Wien, Wien (AT)

(21) Appl. No.: 19/487,390

(22) PCT Filed: May 21, 2024
(86) PCT No.: PCT/IB2024/054916

§ 371 Date: Nov. 24, 2025

(30) Foreign Application Priority Data
May 26, 2023 (IT) 102023000010689

(51) Int. Cl. G09B 23/30; A61B 34/10; A61B 90/00; G09B 23/34

(52) U.S. Cl. CPC G09B 23/303; A61B 34/10; A61B 90/361; G09B 23/34; A61B 2090/365

(57) ABSTRACT

The object of the present invention is a surgical simulator (1) for training and preoperative planning of a surgical operation, such as aneurysm clipping and/or endovascular treatment, comprising: —a support (4) comprising a support body (5) externally delimited by a support external surface (6) having a paradigmatic shape of a body portion of a patient, such as a paradigmatic shape of a skull, so that the support (4) is clampable on the support external surface (6) to simulate a positioning phase of preoperative planning, wherein the support (4) delimits at least a cavity (7, 9) in the support body (6) and a respective cavity aperture (8, 10) on the support external surface (6) for accessing to the respective cavity (7, 9); —at least an anatomical simulator (2, 2′, 3, 3′) that is supported by the support (4), wherein a first anatomical simulator (2, 2′,) of the at least an anatomical simulator (2, 2′, 3, 3′) is removably housed into a first cavity (7) of the at least a cavity (7,9), wherein each anatomical simulator (2, 2′, 3, 3′) comprises at least a respective vascular phantom (11) and a respective container (12) configured to support the vascular phantom (11) , wherein the vascular phantom (11) of each anatomical simulator (2, 2′, 3, 3′) comprises a vascular wall (13) delimiting an hollow structure, the vascular wall (13) being 3D printed in order to replicate a patient-specific vascular structure or a modelled vascular structure.

### FIELD OF THE INVENTION

The present invention refers to a surgical simulator, a kit and a system for training and preoperative planning of a surgical operation, such as aneurysm clipping and/or endovascular treatment.

### STATE OF ART

Modern medicine is based on evidence according to the “Evidence based” paradigm. It means that the safest decision to achieve the best treatment outcome is taken by clinicians considering best available evidence, clinical expertise and patient specificity. When the treatment involves the use of invasive tools, surgeon's experience and good preoperative planning are crucial. Medical knowledge is both theoretical and practical, with the latter being especially crucial in surgery. Surgical experience is acquired with hands-on practice on either patient, which is essential, or simulators, which may be either animal or human corpses, or physical simulators.

Known physical simulators comprise a geometrical three dimensional support and an anatomical simulator which comprises at least a physical reproduction of anatomical models, such as vessels, nerves or tissues. Usually, the support is shaped as a parallelepiped or a cube, defining a cavity into which is inserted the physical reproduction of anatomical models. Usually, the physical reproduction of anatomical models such as vessels reproductions are made by means of casting method, or coating methods of a sacrificial core, which, on the one hand, make the cost of production and time of production, too high, and on the other hand makes the structural properties of physical reproduction of anatomical models not highly realistic nor patient specific.

Therefore, known physical simulators make the learning curve of residents extremely long. Further known simulators, not being sufficiently realistic, cannot be used for preoperative planning of the surgical operation for a specific patient.

Therefore, the need is strongly felt in the field to provide surgical simulators that let to reproduce highly realistic surgical conditions, in terms of training and preoperative planning.

Therefore, the need is strongly felt in the field to provide surgical simulators that let to decrease the time of productions and cost of productions, providing in short time patient specific physical reproduction of anatomical models.

Therefore, the problem underlying the present invention is to provide a surgical simulator, a kit and a system, which have structural and functional features such as to satisfy the aforementioned needs and, at the same time, solve the drawbacks mentioned with reference to the prior art and satisfy the aforesaid felt needs.

### SOLUTION

The aim of the present invention is to provide a surgical simulator, a kit and a system for training and preoperative planning of a surgical operation, such as aneurysm clipping and/or endovascular treatment.

These and other objects and advantages are achieved with a surgical simulator, a kit and a system according to the independent claims.

Some advantageous embodiments are the object of the dependent claims.

According to an aspect, by virtue of the proposed solutions, it is possible to enhance the realism of the surgical simulator, allowing to simulate patient-specific surgical conditions, and the possibility of changing between different cases while keeping the support fixed allows to do it without interruptions.

According to another aspect, by virtue of the proposed solutions, it is possible to enhance the volumes of training using a single surgical simulator, by interchangeable anatomical simulators and/or interchangeable covers.

According to another aspect, by virtue of the proposed solutions, it is possible to provide a surgical simulator which can be reused several times during a training session, also by various users, and it is possible to manipulate, for example by clipping, several times the patient specific vascular phantom without modifications or structural variations to the vascular phantom, making possible to try and select the best surgical strategy, for example clipping strategy, in a wide range of surgical instruments available, such as clip types.

According to another aspect, by virtue of the proposed solutions, it is possible to iteratively use the surgical simulator to identify one potential correct and satisfying procedure setup/plan.

### FIGURE

Further features and advantages of the surgical simulator, the kit and the system, will be apparent from the following description of preferred and nonlimiting embodiments thereof, with reference to the accompanying drawings, in which:

FIG. 1A shows in a first perspective view a surgical simulator according to the present invention, wherein a support and an interchangeable first anatomical simulator are connected, the support having a paradigmatic shape of a skull;

FIG. 1B shows in a second perspective view, opposite to the first perspective view of FIG. 1A, the surgical simulator shown in FIG. 1A, wherein an interchangeable second anatomical simulator which is connected to the support is shown;

FIG. 2 shows in a perspective view, a kit comprising the surgical simulator of FIGS. 1A-1B; wherein the kit may comprise a plurality of interchangeable first anatomical simulators configured to be interchangeably connected to the support by insertion in a first cavity, and/or a plurality of interchangeable second anatomical simulators configured to be interchangeably connected to the support by insertion in a second cavity, and/or a plurality of interchangeable first cavity cover configured to be interchangeably connected to the support or the container of each of the plurality of interchangeable first anatomical simulator, and/or a plurality of interchangeable second cavity cover configured to be interchangeably connected to the support or the container of each of the plurality of interchangeable second anatomical simulator;

FIGS. 3 and 4 show in a lateral view, a surgical simulator according to the present invention, wherein are shown two first cavity covers delimiting each a different first cavity window, which simulates different craniotomies in order to have different limited access to the anatomical simulator using the same support and changing only the first cavity cover;

FIG. 5 shows in a perspective view, a surgical simulator according to the present invention, wherein the support, the container and the vascular phantom are shown in an exploded view;

FIG. 6 shows in a perspective view, an anatomical simulator of the surgical simulator according to the present invention, comprising a container and a vascular phantom having a vascular wall realized by 3D printing reproducing a hollow structure patient specific, which is connectable to a perfusion system;

FIG. 7A shows in a schematic way a system according to the present invention, comprising the surgical simulator of FIGS. 1A-1B or FIG. 5 connected to a perfusion system;

FIG. 7B shows the system of FIG. 7A wherein a catheter is inserted into one of the hydraulic connections of the perfusion system by a Y connector in order to simulate an endovascular surgical operation into the vascular phantom of the surgical simulator.

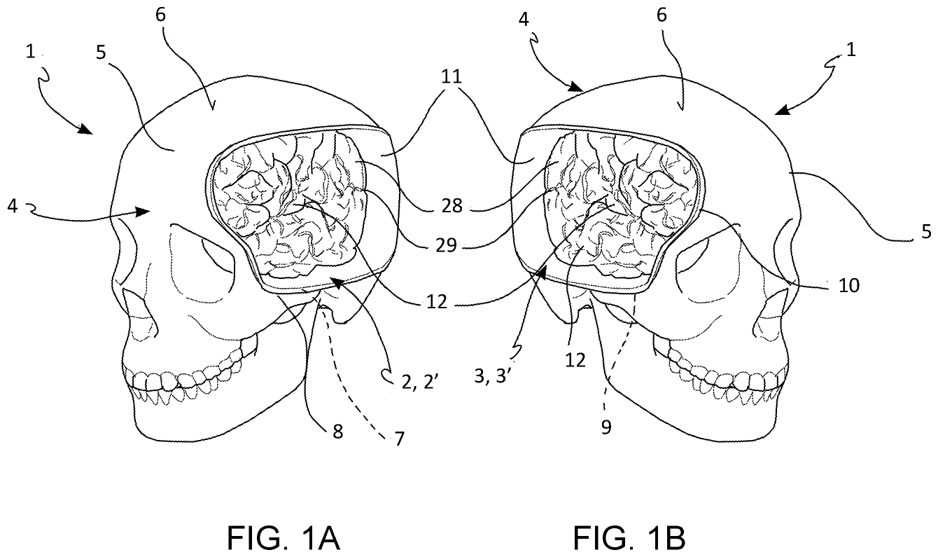

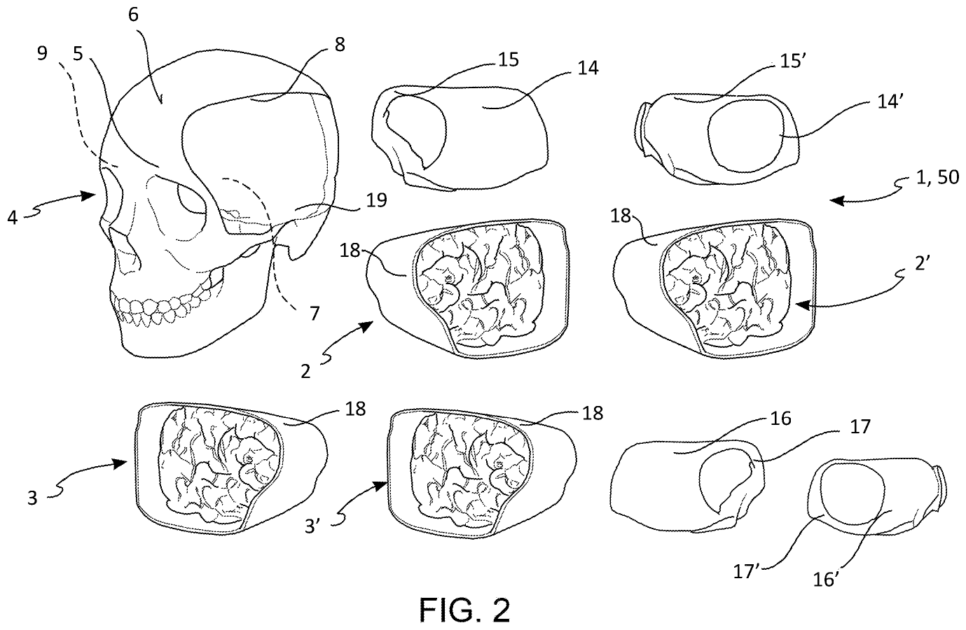

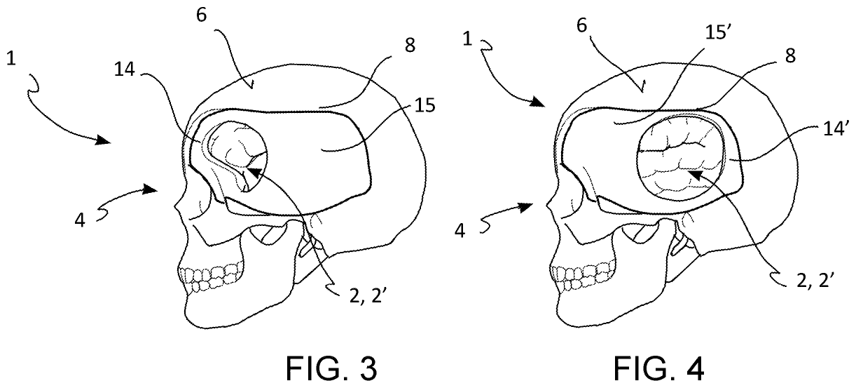

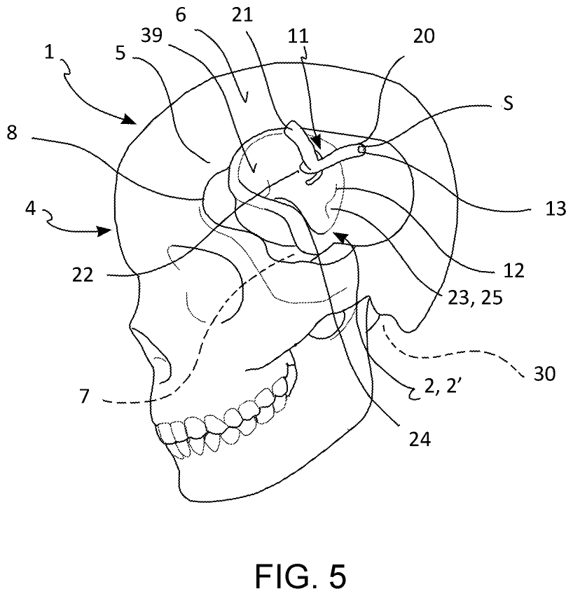

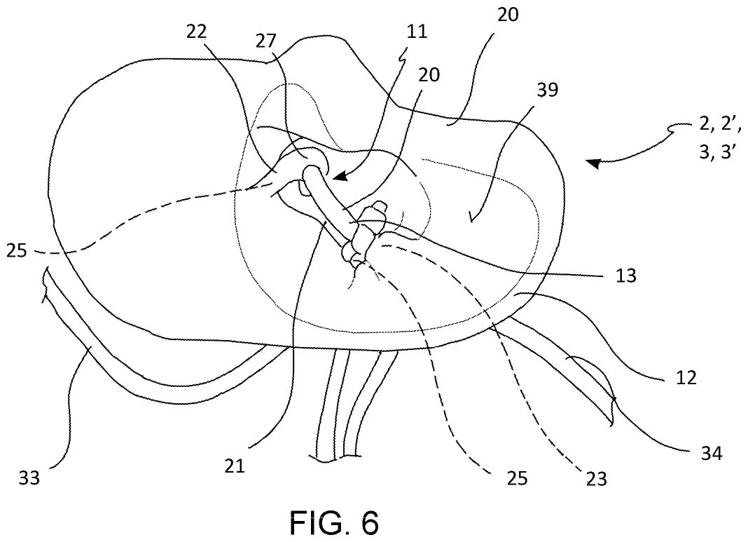

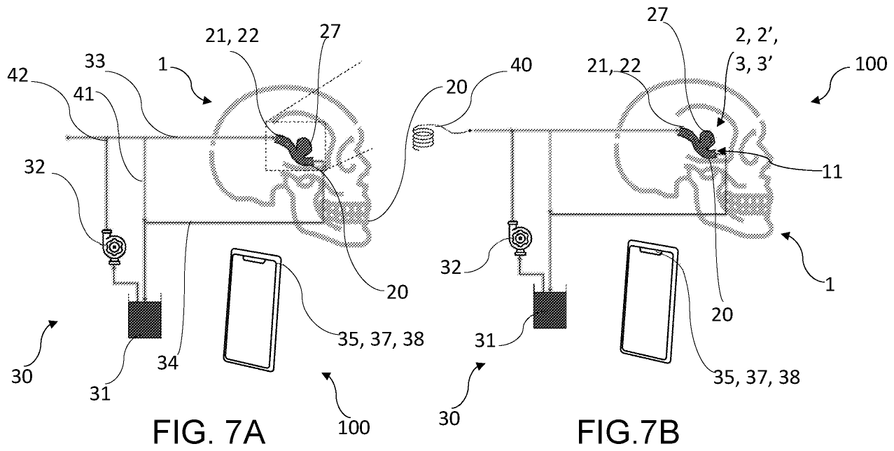

### DESCRIPTION OF SOME PREFERRED EMBODIMENTS

According to a general embodiment, a surgical simulator for training and preoperative planning of a surgical operation, such as aneurysm clipping and/or endovascular treatment and/or vascular endarterectomy, is indicated by reference number 1. Other surgical operations that may be trained and planned by the surgical simulator are surgical bypass for aneurysms, carotid endarterectomy, heart surgery in endovascular and open chest configurations. According to an embodiment, the surgical simulator is a neurosurgical simulator.

The surgical simulator 1 comprises a support 4 and at least an anatomical simulator 2, 2′, 3, 3′ that is supported by the support 4.

The support 4 comprises a support body 5 externally delimited by a support external surface 6. The support external surface 6 has a paradigmatic shape of a body portion of a patient so that the support 4 is clampable on the support external surface 6 to simulate a positioning phase of preoperative planning.

The support 4 delimits at least a cavity 7, 9 in the support body 6 and a respective cavity aperture 8, 10 on the support external surface 6 for accessing to the respective cavity 7, 9.

The surgical simulator 1 comprises a first anatomical simulator 2, 2′, of the at least an anatomical simulator 2, 2′, 3, 3′ that is removably housed into a first cavity 7 of the at least a cavity 7,9 delimited in the support 4.

Each anatomical simulator 2, 2′, 3, 3′ comprises at least a respective vascular phantom 11 and a respective container 12 configured to support at least the respective vascular phantom 11. The vascular phantom 11 of each anatomical simulator 2, 2′, 3, 3′ comprises a vascular wall 13 that delimits a hollow structure.

The vascular wall 13 is 3D printed and/or made by a stretchable, flexible, 3D printed elastomeric material in order to replicate a patient-specific vascular structure or a modelled vascular structure.

In an embodiment, each vascular phantom 11 and/or the vascular wall 13, is elastically deformable so as to be manipulated, for example pinched by a surgical clip, a plurality of times during one or more training and/or planning of the surgical operation without damaging.

In an embodiment, the vascular wall 13 is defined by a single body in a single piece made by additive manufacturing.

By virtue of the additive manufacturing vascular wall 13, it is possible to replicate patient-specific vascular structure or modelled vascular structure, with high precision, high control of the thickness of the vascular wall 13, in much shorter production time then known casting or coating methods, and at lower production costs.

In an embodiment, each anatomical simulator 2, 2′, 3, 3′ comprises also at least a tissue phantom 28, such as a brain phantom, and/or at least a nerve phantom 29. In an embodiment, the vascular phantom 11 and/or the tissue phantom 29 and/or the nerve phantom 29 are obtained from Magnetic Resonance Imaging (MRI), Digital Subtraction Angiography (DSA) or computed tomography (CT) images segmentations patient specific or modelled and adapted to the support 4.

In an embodiment, said paradigmatic shape is the shape of a skull and/or a neck or a chest or an arm or a leg. In the case of the skull paradigmatic shape, a pragmatic geometry of 20 cm×27.1 cm×27.9 cm may be used.

In an embodiment, the support 4 is realized using a geometry of an anatomical portion of the patient or a model, such as an extended craniotomy in a scale 1 to 1 of a patient, or of a 3D model. The support 4 is then realized based on said geometry and printed, for example by additive manufacturing, in a rigid material.

In an embodiment, the surgical simulator 1 comprises a first cavity cover 14, 14′ configured to cover the first cavity aperture 8 of the first cavity 7. In an embodiment, the first cavity cover 14, 14′ is releasably connected to the support 4, preferably by shape coupling with the perimeter of the support body 5 that delimits the first cavity aperture. In an embodiment, the first cavity cover 14, 14′ is releasably connected to the container 12 of the at least a first anatomical simulator 2, 2′, for example by shape coupling with the internal perimeter of the container 12.

In an embodiment, the first cavity cover 14, 14′ delimits at least a first cavity window 15 configured to limit an access to the first cavity 7 housing the at least a first anatomical simulator 2, 2′ for simulating surgical operation conditions. In an embodiment, the first cavity cover 14, 14′, when connected to the support 3 and/or the container 12, is configured to seamlessly continue with the paradigmatic shape of the support external surface 6.

In an embodiment, the support 4 delimits in the support body 5 at least a second cavity 9 and on the support external surface 6 a respective second cavity aperture 10 for accessing to the second cavity 9. In an embodiment, the support 4 may have two cavities, one right and one left.

In an embodiment, the at least a second cavity 9 is configured to house the first anatomical simulator 2, 2′ or a second anatomical simulator 3, 3′ of the at least an anatomical simulator 2, 2′, 3, 3′.

In an embodiment, the support 4 is orientable in the space without any base fixed to it, to maintain a specific position on a supporting surface, such an operating table. In other words, the support 4 is free to be clamped on a operating table or on a clamping system that is used during surgical operations, in any operating position. In an alternative embodiment, the support 4 is fixable on a base by fixing means.

In an embodiment, the surgical simulator 1 comprises a plurality of said at least an anatomical simulator 2, 2′, 3, 3′ comprising said first anatomical simulator 2, 2′ and at least a second anatomical simulator 3, 3′.

In an embodiment, the second anatomical simulator 3, 3′ is removably housed into the second cavity 9.

In an embodiment, the vascular phantom 11 of the first anatomical simulator 2, 2′ and the vascular phantom 11 of the second anatomical simulator 3, 3′ are distinct from each other so as to simulate different surgical operations on different cavities 7, 9 of the same support 3.

By virtue of the provision of a support 4 having paradigmatic external shape and at least two different cavities 7, 9, or even more, it is possible to train on the same support 4, different cases depending on the interchangeable anatomical simulator 2, 2′, 3, 3′ used, and with different surgical approaches.

In an embodiment, the surgical simulator 1 comprises a second cavity cover 16, 16′ configured to cover the second cavity aperture 10, wherein the second cavity cover 16, 16′ is releasably connected to the support 3 and/or to the container 12 of the second anatomical simulator 3, 3′. In an embodiment, the second cavity cover 16, 16′, when connected to the support 3, is configured to seamlessly continue with the paradigmatic shape of the support external surface 6.

In an embodiment, the second cavity cover 16, 16′ delimits at least a second cavity window 17 configured to limit an access to the second cavity 9 housing the second anatomical simulator 3, 3′ for simulating surgical operation conditions.

By virtue of such first cavity cover 14, 14′ and/or second cavity cover 16, 16′ it is possible to limit the access to the anatomical simulator, and in particular to the vascular phantom, by simulating, in case of a skull support, a craniotomy (restricted surgical access), and it is also possible using the same support to train or planning different cases, being the first cavity cover 14, 14′ and/or second cavity cover 16, 16′ interchangeable with another cover having a different respective first cavity window 15 and/or different second cavity window 16 simulating different craniotomy.

By virtue of such first cavity cover 14, 14′ and/or second cavity cover 16, 16′, during training, it is possible to simulate different accesses (craniotomies locations) for different cases, for example different aneurysm cases.

By virtue of such first cavity cover 14, 14′ and/or second cavity cover 16, 16′, during preoperative planning, the cover may replicate a patient-specific craniotomy specifically designed for that patient.

In an embodiment, the container 12 of each anatomical simulator 2, 2′, 3, 3′ is conformed to couple with the support body 5 in the respective cavity 7, 9 leaving a free access to the respective vascular phantom 11.

In an embodiment, wherein the container 12 of each anatomical simulator 2, 2′, 3, 3′ comprises a respective container connecting portion 18 configured to couple with a corresponding support connecting portion 19 of the support body 5 by interference and/or shape or fit coupling. In an embodiment, the support connecting portion 19 is a portion of the support body 5 close to the respective aperture 8, 10 on the support external surface 6, which delimits around a perimeter the respective cavity 7,9. In an embodiment, container connecting portion 18 is externally counter shaped to the support connecting portion 19 so that to easily match without error by a user interchanging one anatomical simulator 2, 3 with another anatomical simulator 2′, 3′ that is configured to be coupled to the same cavity 7, 9.

In an embodiment, the container 12 of each anatomical simulator 2, 2′, 3, 3′ is cup shaped. In an embodiment, the container 12 of each anatomical simulator 2, 2′, 3, 3′is tapered for facilitating the insertion of the container in the respective cavity 7, 9. For Example the container 12 may be cup shaped, having tapered conical surface externally. In an embodiment the container 12 of each anatomical simulator 2, 2′, 3, 3′ delimits with a container internal surface 39, an internal volume configured to accommodate the vascular phantom 11 and/or the tissue phantom 29 and/or the nerve phantom 29.

In an embodiment, the vascular wall 13 of the vascular phantom 11 of each anatomical simulator 2, 2′, 3, 3′ delimits at least first tube 20 and at least a second tube 21 fluidically connected to each other so that each vascular phantom 11 is fluidically connectable to a perfusion system 30.

In an embodiment, each first tube 20 is fluid tight connected to a respective first hole 23 passing through the respective container 12. In an embodiment each second tube 21 is fluid tight connected to a respective second hole 24 passing through the respective container 12. In an embodiment, the provision of the respective holes 23, 24, 25 passing through the container 12 avoids hydraulic connections of the perfusion system 30 to interfere with the respective cavity aperture 8, 10, simulating the real operative conditions around the support 4. In an embodiment, the support 4 comprises at least a perfusion aperture 26 passing through the support body 5 in communication with said at least a cavity 7, 9.

In an embodiment, the vascular wall 13 of the vascular phantom 11 of each anatomical simulator 2, 2′, 3, 3′ delimits at least third tube or bypass tube 22 fluidically connected to at least one or either the respective first tube 20 and the respective second tube 21. In an embodiment, the third tube 22 or the second tube 21 is configured to receive a catheter 40, which may be used for surgical operation into the vascular phantom 11. In an embodiment, the third tube 22 or the second tube 21 is configured to receive a fluorophore injection, so that by an IR camera is possible to detect if the simulated operation on the vascular phantom 11 has been completed. In an embodiment, each third tube 22 is fluid tight connected to a respective third hole 25 passing through the respective container 12.

In an embodiment, the container 12 comprises at least an internal hydraulic connector that protrudes from the container internal surface 39 and ends delimiting the respective hole 23, 24, 25. In this way, it is facilitated the connection of the anatomical phantom 11 to the container 12 in a sealed or fluid tight way to the respective at least an internal hydraulic connector or to the hydraulic connections of the perfusion system 30, for example by glue or cable ties. In this way, it is further facilitated the fluid tight connection of the hydraulic connections of the perfusion system 30, through said respective holes 23, 24, 25 passing from an external surface of the container 12.

In an embodiment, the vascular wall 13 of the vascular phantom 11 of each anatomical simulator 2, 2′, 3, 3′ has a wall thickness S. In an embodiment the wall thickness S is between 0.05 mm and 3.00 mm. In an embodiment the wall thickness S is between 0.20 mm and 3.00 mm. In an embodiment the wall thickness S is between 0.20 mm and 0,60 mm. In an embodiment the wall thickness S is between 0.20 mm and 0.50 mm. In an embodiment, the vascular wall 13 of the vascular phantom 11 of at least one anatomical simulator 2, 2′, 3, 3′ is coated and or painted with an external layer.

In an embodiment, the vascular wall 13 of the vascular phantom 11 of each anatomical simulator 2, 2′, 3, 3′ comprises at least a dilatation portion 27 configured to replicate a patient specific aneurysm or a modelled aneurysm.

In an embodiment, the wall thickness S along a respective development direction around which the vascular wall 13 of the vascular phantom 11 of each anatomical simulator 2, 2′, 3, 3′ is developed, is constant or variable depending on the patient-specific vascular structure or the modelled vascular structure.

In an embodiment, the vascular wall 13 of the vascular phantom 11 of each anatomical simulator 2, 2′, 3, 3′ is realized in a silicone material. In an embodiment, the silicone material is transparent.

In an embodiment, the vascular wall 13 of the vascular phantom 11 of each anatomical simulator 2, 2′, 3, 3′ is realized in a transparent material.

In an embodiment, the vascular wall 13 of the vascular phantom 11 of each anatomical simulator 2, 2′, 3, 3′ is realized in 100 % pure silicone. For example, the silicone is a TrueSil 20A of SpectroPlast, Zurich Switzerland.

In an embodiment, the vascular wall 13 of the vascular phantom 11 of each anatomical simulator 2, 2′, 3, 3′ is a material, preferably silicone based, having an elongation at break between 50% and 1000%, preferably between 50% and 500%, measured according to iso 37 type 4 standard.

In an embodiment, the vascular wall 13 of the vascular phantom 11 of each anatomical simulator 2, 2′, 3, 3′ is an elastic material, preferably silicone based, having a tear resistance of at least 0.1 N/mm, and preferably inferior to 10.0 N/mm, measured according to ASTM D624 Type C standard. In an embodiment the tear resistance of the elastic material is comprised between 1 N/mm2 and 6N/mm2, or between 3N/mm2 and 6N/mm2.

In an embodiment, the vascular wall 13 of the vascular phantom 11 of each anatomical simulator 2, 2′, 3, 3′ is an elastic material, preferably silicone based, having a Shore A hardness inferior to 25 A, preferably between 10 A and 20 A, measured according to ISO7619-1 standard.

In an embodiment, the vascular wall 13 of the vascular phantom 11 of each anatomical simulator 2, 2′, 3, 3′ is an elastic material, preferably silicone based, having a tensile strength between 3N/mm2 and 6N/mm2, measured according to ISO 37 Type 4 standard.

In an embodiment, each vascular phantom 11 of each anatomical simulator 2, 2′, 3, 3′ is made in silicone by silicone additive manufacturing technology, using for example a method as disclosed in document WO2022008721, and for example by using silicone 3d printing ink disclosed in document WO2019053258.

By virtue of the material of the vascular wall 13 realized by 3D additive manufacturing, it is possible to obtain vascular phantoms 11 that are highly realistic in terms of mechanical properties, dimensioning and haptic feedback.

By virtue of the material of the vascular wall 13 realized by 3D additive manufacturing, it is possible to obtain vascular phantoms 11 that are repeatedly pinchable by surgical clips more than 180 times, without suffering of any modification.

In an embodiment, the container 12 of each anatomical simulator 2, 2′, 3, 3′ is at least partially elastically deformable and/or stretchable and flexible, for example in a region where the respective vascular phantom 11 is supported, so that to simulate deformations of soft tissues during surgical operation.

In an embodiment, the container 12 of each anatomical simulator 2, 2′, 3, 3′ is made at least partially of an elastomer, such as a silicone elastomer or polyurethane elastomer, in order to elastically stretch when the respective vascular phantom 11 supported by the container 12 is manipulated.

In an embodiment, the container 12 of each anatomical simulator 2, 2′, 3, 3′, is fully made in elastically deformable material. In an embodiment, the container 12 of each anatomical simulator 2, 2′, 3, 3′ is made by 3D printing or by injection molding. In an embodiment, the container 12 of each anatomical simulator 2, 2′, 3, 3′ is made by a stretchable, flexible, elastomeric material.

In an embodiment, a portion of the container follows the perimeter of the respective cavity 7, 9 in correspondence to the respective aperture 8, 10, and said portion is disposed and compressed between the support body 5 and the respective cover of cavity 15, 15′, 16, 16′.

By virtue of the stretchable and flexible container 12, it is possible to, in contrast with rigid containers, simulate surgical conditions of the anatomical part to which the vascular phantom is attached, so that when manipulating the vascular phantom it is possible to simulate also the deformation of soft tissues.

The present invention also relates to a kit 50 for training and preoperative planning of a surgical operation.

The kit 50 comprises a surgical simulator 1 according to any of the described embodiments. The kit 50 further comprises a plurality of said first anatomical simulator 2, 2′, wherein each of the first anatomical simulators 2, 2′ is interchangeably connectable to said support 4 into said first cavity 7 with respect to another first anatomical simulator 2, 2′.

In an embodiment, the kit 50 comprises a plurality of said second anatomical simulator 3, 3′, wherein each of the second anatomical simulators 3, 3′ is interchangeably connectable to said support 4 into said second cavity 9 with respect to another second anatomical simulator 3, 3′.

By virtue of the provision of the support 4 having one or more cavities 7, 9 and a plurality of first interchangeable anatomical simulators 2, 2′ and/or a plurality of second interchangeable anatomical simulators 3, 3′, it is possible to enhance a number of the training and preoperative planning sessions that may be done by trainers, speeding up the preparation of the surgical simulator.

In an embodiment, said kit 50 comprises a plurality of said first cavity cover 14, 14′, wherein each of said first cavity cover 14, 14′ is interchangeably connectable to said support 4 covering said first cavity 7 or to each first anatomical simulator 2, 2′; wherein each of the first cavity cover 14, 14′ delimits a respective first cavity window 15 in different positions so as to simulate a variety of surgical limited access to the first anatomical simulator 2, 2′ using the same support 4, In an embodiment.

In an embodiment, wherein said kit 50 comprises a plurality of said second cavity cover 16, 16′, wherein each of said second cavity cover 16, 16′ is interchangeably connectable to said support 4 covering said second cavity 9 or to each second anatomical simulator 3, 3′; wherein each of the second cavity cover 16, 16′ delimits a respective second cavity window 17 in different positions so as to simulate a variety of surgical limited access to the second anatomical simulator 3, 3′ using the same support 4.

By virtue of the provision of the support 4, the plurality of interchangeable first cavity cover 14, 14′ having different first cavity windows 15, and/or the plurality of interchangeable second cavity cover 16, 16′ having different second cavity windows 17, it is possible to face different surgical procedures and approaches using the same support 4, even maintaining the support 4 fixed on a clamping system, by only changing one of the plurality of interchangeable first cavity cover 14, 14′ or one of the plurality of interchangeable second cavity cover 16, 16′.

The present invention also relates to a system 100 for training and preoperative planning of a surgical operation.

The system 100 comprises a surgical simulator 1 according to any of the described embodiments or a kit 50 according to any of the described embodiments.

The system 100 comprises a perfusion system 30 comprising a tank 31 suitable for containing a fluid, for example the fluid being suitable for simulating blood, a pump 32 in hydraulic connection with the tank 31, at least a first hydraulic connection 33 configured for fluidically connecting the tank 31 to the vascular phantom 11 of the at least an anatomical simulator 2, 2′, 3, 3′, a second hydraulic connection 34 configured to fluidically connect the pump 32 to the vascular phantom 11 of the at least an anatomical simulator 2, 2′, 3, 3′. In an embodiment, the fluid is a blood like liquid or a liquid simulating blood at least in the color, so that can be seen passing through the perfused vascular phantom 11 having the vascular wall 13 transparent. In an embodiment, the pump 32 is configured to pump the fluid from the tank to the vascular phantom 11 through the first hydraulic connection 33.

In an embodiment, the perfusion system 30 comprises a collateral hydraulic connection 41 the put in communication the first hydraulic connection 33 and second hydraulic connection 34, so that when the flow through the vascular phantom 11 is obstructed, the perfusion system can work anyways. In an embodiment, the perfusion system 30 comprises an Y connector 42 which is connected between the pump and the vascular phantom 11, preferably on the first hydraulic connection 33, so that a catheter 40 may be inserted in the vascular phantom 11 or a fluorophore solution may be injected toward the vascular phantom 11.

In an embodiment, the system 100 comprises at least one camera having a camera 3D sensor 35 configured to record 3D videos. In an embodiment, the camera 3D sensor 35 allows to record not only the normal procedure simulated, but also the position in space of the tools and movements in relation to the simulated anatomy. The 3D video can then be used for a number of reasons, for example it can represent the real plan of the procedure to replicate intraoperatively, or it can be used to do computations about the training session.

In an embodiment, the system 100 comprises an augmented reality system 36, for example a smart phone connected to a server, configured to recognize the support 4 and/or the anatomical simulator 2, 2′, 3, 3′ so as to match it to a known model and superimposing additional information on top of it, such as a superimposed virtual reality layer.

In an embodiment, wherein the system 100 comprises a leak detection sensor 36 configured to detect a rupture of the vascular phantom 11 of the respective anatomical simulator 2, 2′, 3, 3′, said leak detection sensor 36 being for example a pressure sensor connected to the perfusion system 30.

In an embodiment, wherein the system 100 comprises an infrared image acquisition sensor 37.

In an embodiment, the system 100 comprises a position sensor 38 configure to extract at least one position from the system selected from: a clip configured to be applied to the at least an anatomical simulator 2, 2′, 3, 3′, a surgical instrument.

In an embodiment, the system 100 comprises an automatic surgical planner for the definition of key positions/3D configurations of tools and anatomy, and/or a target surgical setup to preselect tools.

In an embodiment, wherein the system 100 comprises a sensor mounted in the anatomical phantom 2, 2′, 3, 3′ or on the support 4 or in the at least one camera, sending data to an analyser, the analyser sending a response to a visualization system, wherein the sensor can a pressure sensor at the inlet/outlet that measures pressure in real time and sends it to a computer where a real time simulation of the vessel is running, with the pressures applied in the simulation equal to the pressures measured on the vascular phantom 11, or wherein the sensor can be a 3D camera recognizing the vascular phantom 11, sending a 3D model of the phantom to the computer where a simulation is running: such a simulation can for example compute the strains and the stresses of the model in real time and show where the model has high strains/stresses. A simpler example of such a sensor can come from the use of two flow meters at the inlet and outlet of the vascular phantom 11 and send the data to a computer that elaborates the data and detects when a clip is applied on the dilatation portion 27 of the vascular phantom 11.

In an embodiment, the system 100 comprises a clamping device configured to clamp the support 4 during the training and preoperative planning of the surgical operation.

In an embodiment, the system 100 comprises a microscope.

The present invention also relates to a method of realization of a surgical simulator 1 for training and preoperative planning of a surgical operation.

The method comprises the steps of:
3D modelling a modelled hollow structure replicating a patient specific vascular structure,
providing a support 4 comprising a support body 5 externally delimited by a support external surface 6 having a paradigmatic shape of a body portion of a patient, such as a paradigmatic shape of a skull, so that the support 4 is clampable on the support external surface 6 to simulate a positioning phase of preoperative planning, wherein the support 4 delimits at least a cavity 7, 9 in the support body 6 and a respective cavity aperture 8, 10 on the support external surface 6 for accessing to the respective cavity 7, 9,
manufacturing by 3D printing a vascular wall 13 delimiting a hollow structure that replicates the modelled hollow structure, the 3D printed vascular wall being stretchable and flexible,
providing a container 12 configured to be housed into a first cavity 7 or a second cavity 9 of the at least a cavity 7,9 and configured to support the vascular wall 13,
assembling the vascular wall 13 in the container 12 obtaining at least an anatomical simulator 2, 2′, 3, 3′,
assembling the at least an anatomical simulator 2, 2′, 3, 3′ and the support 4, by inserting the container 12 into the at least a cavity 7,9 obtaining the surgical simulator.

In an operating mode, the surgical simulator 1 is according to any of the previous described embodiments. In an operative mode, the 3D modelling phase is obtained from Magnetic Resonance Imaging (MRI), Digital Subtraction Angiography (DSA) or computed tomography (CT) patient specific images segmentations.

Thanks to the disclosed embodiments, it is possible to provide a surgical simulator that can be used for training, allowing high volumes of training with a single platform.

Thanks to the disclosed embodiments, it is possible to provide a surgical simulator that allow to simulate both endovascular treatment and clipping treatment.

Thanks to the disclosed embodiments, it is possible to provide a surgical simulator suitable to perform preoperative planning and simulate the surgery before actually doing it because of the support having an anatomical external geometry simulating the actual encumbers of a patient, and thanks to 3D printed hollow structures of the vascular phantoms, which are highly realistic and elastically deformable more than 100 hundred times without damages, so that a plurality of attempts during training of clipping the vascular phantoms are possible until the best approach is found.

Thanks to the disclosed embodiments, it is possible to provide a surgical simulator is effective in training different skills, in particular in reducing operative time, improve instruments selection, such as aneurysm clips, and reduce implantation attempts.

### REFERENCE LIST

1 Surgical simulator
2, 2′ first anatomical simulator
3, 3′ second anatomical simulator
4 support
5 support body
6 support external surface
7 first cavity
8 first cavity aperture
9 second cavity
10 second cavity aperture
11 vascular phantom
12 container
13 vascular wall
14, 14′ first cavity cover
15, 15′ first cavity window
16, 16′ second cavity cover
17, 17′ second cavity window
18 container connecting portion
19 support connecting portion
20 first tube
21 second tube
22 third tube
23 first hole
24 second hole
25 third hole
26 perfusion aperture
27 dilatation portion
28 tissue phantom
29 nerve phantom
30 perfusion system
31 tank
32 pump
33 first hydraulic connection
34 second hydraulic connection
35 camera sensor
36 leak detection sensor
37 infrared sensor
38 position sensor
39 container internal surface
40 catheter
41 collateral hydraulic connection
42 Y connector
50 kit
100 system

1. A surgical simulator for training and preoperative planning of a surgical operation, such as aneurysm clipping and/or endovascular treatment and/or vascular endarterectomy, comprising:
a support comprising a support body externally delimited by a support external surface having a paradigmatic shape of a body portion of a patient, such as a paradigmatic shape of a skull, so that the support is clampable on the support external surface to simulate a positioning phase of preoperative planning, wherein the support delimits at least a cavity in the support body and a respective cavity aperture on the support external surface for accessing to the respective cavity;
at least an anatomical simulator that is supported by the support, wherein a first anatomical simulator of the at least an anatomical simulator is removably housed into a first cavity of the at least a cavity, wherein each anatomical simulator comprises at least a respective vascular phantom and a respective container configured to support the at least a respective vascular phantom, wherein the at least a respective vascular phantom of each anatomical simulator comprises a vascular wall delimiting an hollow structure, the vascular wall being 3D printed in order to replicate a patient-specific vascular structure or a modelled vascular structure.

2. The surgical simulator according to claim 1, comprising a first cavity cover configured to cover the first cavity aperture of the first cavity, wherein the first cavity cover is releasably connected to the support and/or to the container of the at least a first anatomical simulator,
wherein the first cavity cover delimits at least a first cavity window configured to limit an access to the first cavity housing the at least a first anatomical simulator for simulating surgical operation conditions.

3. The surgical simulator according to claim 1,
wherein the container of each anatomical simulator is conformed to couple with the support body in the respective cavity leaving a free access to the respective vascular phantom,
and/or wherein the container of each anatomical simulator comprises a respective container connecting portion configured to couple with a corresponding support connecting portion of the support body by interference and/or shape or fit coupling,
and/or the container of each anatomical simulator is cup shaped, preferably being tapered for facilitating the insertion of the container in the respective cavity.

4. The surgical simulator according to claim 1,
wherein the support delimits in the support body at least a second cavity and on the support external surface a respective second cavity aperture for accessing to the second cavity,
wherein the at least a second cavity is configured to house the first anatomical simulator or a second anatomical simulator of the at least an anatomical simulator, and/or wherein the first cavity cover, when connected to the support and/or the container, is configured to seamlessly continue with the paradigmatic shape of the support external surface.

5. The surgical simulator according to claim 4, comprising a plurality of said at least an anatomical simulator comprising said first anatomical simulator and at least a second anatomical simulator,
wherein the second anatomical simulator is removably housed into the second cavity, wherein the vascular phantom of the first anatomical simulator and the vascular phantom of the second anatomical simulator are distinct from each other so as to simulate different surgical operations on different cavities of the same support

6. The surgical simulator according to claim 4, comprising a second cavity cover configured to cover the second cavity aperture wherein the second cavity cover is releasably connected to the support and/or to the container of the second anatomical simulator,
wherein the second cavity cover delimits at least a second cavity window configured to limit an access to the second cavity housing the second anatomical simulator for simulating surgical operation conditions.

7. The surgical simulator according to claim 1,
wherein the vascular wall of the vascular phantom of each anatomical simulator delimits at least first tube and at least a second tube fluidically connected to each other so that each vascular phantom is fluidically connectable to a perfusion system,
preferably wherein each first tube is fluid tight connected to a respective first hole passing through the respective container, and wherein each second tube is fluid tight connected to a respective second hole passing through the respective container avoiding hydraulic connections of the perfusion system to interfere with the respective cavity aperture, wherein the support comprises at least a perfusion aperture passing through the support body in communication with said at least a cavity,

8. The surgical simulator according to claim 7, wherein the vascular wall of the vascular phantom of each anatomical simulator delimits at least third tube or bypass tube fluidically connected to at least one or either the respective first tube and the respective second tube wherein the third tube is configured to receive a catheter, preferably wherein each third tube is fluid tight connected to a respective third hole passing through the respective container.

9. The surgical simulator according to claim 1, claims, wherein the vascular wall of the vascular phantom of each anatomical simulator has a wall thickness(S) between 0.60 mm0.05 to 3.00 mm; and/or wherein the vascular wall of the vascular phantom of each anatomical simulator comprises at least a dilatation portion configured to replicate a patient specific aneurysm or a modelled aneurysm;
and/or the vascular wall of the vascular phantom of each anatomical simulator is coated and or painted with an external layer.

10. The surgical simulator according to claim 9, wherein the wall thickness along a respective development direction around which the vascular wall of the vascular phantom of each anatomical simulator is developed, is constant or variable depending on the patient-specific vascular structure or the modelled vascular structure.

11. The surgical simulator according to claim 1, wherein the vascular wall is made by a stretchable, flexible, 3D printed elastomeric material,
and/or wherein the vascular wall of the vascular phantom of each anatomical simulator is realized in a silicone material;
and/or wherein the vascular wall of the vascular phantom of each anatomical simulator is realized in 100 % pure silicone;
and/or wherein the vascular wall of the vascular phantom of each anatomical simulator is a material, preferably silicone based, having an elongation at break between 50% and 1000%, preferably between 50% and 500%;
and/or wherein the vascular wall of the vascular phantom of each anatomical simulator is an elastic material, preferably silicone based, having a tear resistance of at least 1 N/mm, and preferably inferior to 6.0 N/mm;
and/or wherein the vascular wall of the vascular phantom of each anatomical simulator is an elastic material, preferably silicone based, having a Shore A hardness inferior to 25A, preferably between 10 A and 20 A;
and/or wherein the vascular wall of the vascular phantom of each anatomical simulator is an elastic material, preferably silicone based, having a tensile strength between 0.1 N/mm2 and 10N/mm2;
and/or wherein each vascular phantom of each anatomical simulator is made in silicone by silicone additive manufacturing technology.

12. The surgical simulator according to claim 1, wherein the container of each anatomical simulator at least partially stretchable and flexible, for example in a region where the respective vascular phantom is supported, so that to simulate deformations of soft tissues during surgical operation; and/or wherein
the container of each anatomical simulator is made at least partially of an elastomer, such as a silicone elastomer or polyurethane elastomer, in order to elastically stretch when the respective vascular phantom supported by the container is manipulated.

13. The surgical simulator according to claim 1,
wherein each container is conformed to couple with the support body in the respective cavity leaving a free access to at least a respective vascular phantom. and
wherein each container delimits with a respective container internal surface an internal volume configured to accommodate the at least a respective vascular phantom.

14. The surgical simulator according to claim 13, wherein the container of each anatomical simulator is made at least partially of an elastomer, such as a silicone elastomer or polyurethane elastomer, in order to elastically stretch when the respective vascular phantom supported by the container is manipulated.

15. A kit for training and preoperative planning of a surgical operation, comprising:
a surgical simulator according to claim 1 and
a plurality of said first anatomical simulator, wherein each of the first anatomical simulator is interchangeably connectable to said support (into said first cavity.

16. The kit according to claim 15, comprising:
a plurality of said second anatomical simulator-(3, wherein each of the second anatomical simulator is interchangeably connectable to said support into said second cavity,
and/or wherein said kit comprises a plurality of said first cavity cover wherein each of said first cavity cover is interchangeably connectable to said support covering said first cavity or to each first anatomical simulator wherein each of the first cavity cover delimits a respective first cavity window in different positions so as to simulate a variety of surgical limited access to the first anatomical simulator using the same support,
and/or wherein said kit comprises a plurality of said second cavity cover, wherein each of said second cavity cover is interchangeably connectable to said support covering said second cavity or to each second anatomical simulator; wherein each of the second cavity cover delimits a respective second cavity window in different positions so as to simulate a variety of surgical limited access to the second anatomical simulator using the same support.

17. A system for training and preoperative planning of a surgical operation, comprising:
a surgical simulator according to claim 14;
a perfusion system comprising a tank suitable for containing a fluid, for example the fluid being suitable for simulating blood, a pump in hydraulic connection with the tank, at least a first hydraulic connection configured for fluidically connecting the tank to the vascular phantom of the at least an anatomical simulator, a second hydraulic connection configured to fluidically connect the pump to the vascular phantom of the at least an anatomical simulator.

18. The system according to claim 17, comprising:
at least one camera sensor configured to record 3D video;
and/or wherein the system comprises an augmented reality system, for example a smart phone connected to a server, configured to recognize the support and/or the anatomical simulator so as to match it to a known model and superimposing additional information on top of it, such as a superimposed virtual reality layer;
and/or wherein the system comprises a leak detection sensor configured to detect a rupture of the vascular phantom of the respective anatomical simulator, said leak detection sensor being for example a pressure sensor connected to the perfusion system,
and/or wherein the system comprises an infrared image acquisition sensor
and/or wherein the system comprises a position sensor configure to extract at least one position from the system selected from: a clip configured to be applied to the at least an anatomical simulator, a surgical instrument.
